(19) United States
(12) Patent Application Publication
(10) Pub. No.: US 2016/0082201 A1
(43) Pub. Date: Mar. 24, 2016

(54) SYRINGE INJECTION AID

(71) Applicant: Ralph P. Stillman, Excelsior, MN (US)

(72) Inventor: Ralph P. Stillman, Excelsior, MN (US)

(21) Appl. No.: 14/864,595

(22) Filed: Sep. 24, 2015

Related U.S. Application Data
(60) Provisional application No. 62/054,596, filed on Sep. 24, 2014.

(51) Int. Cl. A61M 5/46

(52) U.S. Cl. CPC A61M 5/46

(57) ABSTRACT

A syringe injection aid apparatus comprises a body having a lower opening defined by an actuation collar, a syringe-interfacing plunger substantially slidably contained within the body, and a locking mechanism pivotally secured to the body. The locking mechanism is configured to prevent forward movement of the syringe-interfacing plunger when in a locked position and to enable forward movement of the syringe-interfacing plunger when in an unlocked position. The unlocked position is activated by external contact with the actuation collar.

### FIELD OF THE DISCLOSURE

The present disclosure relates to injection devices and, more particularly, to an injection aid device configured to interact with a syringe.

### BACKGROUND

Numerous individuals are faced daily with the task of injecting themselves or others with medication. A typical injection scene finds the individual with a two-handed task of holding a vial of medication in one hand while trying to align a syringe, held in the other hand, with the vial. Once the needle of the syringe is injected into the vial, the individual must hold the syringe and vial in position while drawing back the plunger of the syringe to withdraw a specific dosage amount into the syringe. The individual must then position the syringe at a desired injection site then maintain that position and needle depth while simultaneously depressing the syringe plunger to deliver the medication. The multi-step process of delivering an injection requires significant manual dexterity. For those individuals lacking manual dexterity, the process can become very difficult and can present the individual with frustration and even safety issues due to, for example, the dropping and contamination of the syringe, unintended jabs with the needle, and, unfortunately, even the failure to deliver a medication that is needed to maintain health. As such, there is a need for an injection aid that can ensure confident and successful delivery of an injection time after time.

### SUMMARY

A syringe injection aid includes a body, a syringe-interfacing plunger slidably contained within the body, and a locking mechanism. The body has a lower opening defined by an actuation collar. The locking mechanism is pivotally secured to the body and has a locked and unlocked position. The locking mechanism is configured to prevent forward movement of the syringe-interfacing plunger when in a locked position and is configured to enable forward movement, i.e. towards an injection site, of the syringe-interfacing plunger when in an unlocked position. The unlocked position is activated by external contact with the activation collar.

A syringe injection aid includes a containment means, a syringe-interfacing means slidably contained within the containment means and a locking means. The syringe-interfacing means is for interfacing with a syringe having a needle. The locking means is pivotally secured to the containment means and has a locked and unlocked position. The locking means is for preventing forward movement of the syringe-interfacing means when in a locked position and for enabling forward movement, i.e. towards an injection site, of the syringe-interfacing means when in an unlocked position. The actuation means is for unlocking the locking means upon being externally contacted.

A syringe injection aid includes an outer shell, a plunger, and a tube support. The plunger is slidably contained by the outer shell and is configured to interface with a syringe; the plunger has an injection delivery position. The tube support is coupled to the outer shell and includes a pivotable locking arm and an actuation collar. Upon external actuation of the actuation collar the locking arm is moved into an unlocked position enabling the plunger to slide past the locking arm to the injection delivery position. Upon retreat from the injection delivery position, the plunger causes the locking arm to move into a locked position.

An embodiment of a syringe injection aid includes a body, a plunger, and a mechanism. The body is configured to substantially confine a syringe having a syringe plunger, a syringe body, and a needle. The plunger is substantially, slidably contained within the body. The mechanism is configured to interface with a surface of the plunger. Upon the mechanism engaging the surface of the plunger, the mechanism also engages the syringe body to move the needle of the syringe a distance beyond the confines of the body. Upon the mechanism subsequently disengaging the surface of the plunger, the plunger engages the syringe plunger to move the syringe plunger toward the syringe body while maintaining the distance of the needle from the body.

The mechanism may comprise a ratchet for engaging an interior, slatted surface of the plunger. The plunger may include a graspable element that is exterior of the body. The plunger may additionally be configured to activate a position lock and the body may additionally be configured to deactivate the position lock upon the body contacting the neck of a medication vial. The plunger may additionally be configured to draw the syringe plunger away from the syringe body. The body may additionally be configured to establish a dosage for the syringe.

The above summary is not intended to describe each embodiment or every implementation. A more complete understanding will become apparent and appreciated by referring to the following detailed description and claims in conjunction with the accompanying drawings.

### BRIEF DESCRIPTION OF THE DRAWINGS

FIG. 1 is an exploded assembly view of a syringe injection aid according to various embodiments.

FIG. 2 is side perspective of the outer shell of FIG. 1.

FIG. 3 is a substantially assembled syringe injection aid relative to a syringe according to various embodiments.

FIG. 4 is a fully assembled syringe injection aid according to various embodiments.

FIG. 5 is an exploded assembly view of a syringe injection according to various embodiments.

FIG. 6A is an exploded assembly view of a syringe injection according to various embodiments.

FIG. 6B is a substantially assembled syringe injection aid of FIG. 6A relative to a syringe according to various embodiments.

### DETAILED DESCRIPTION

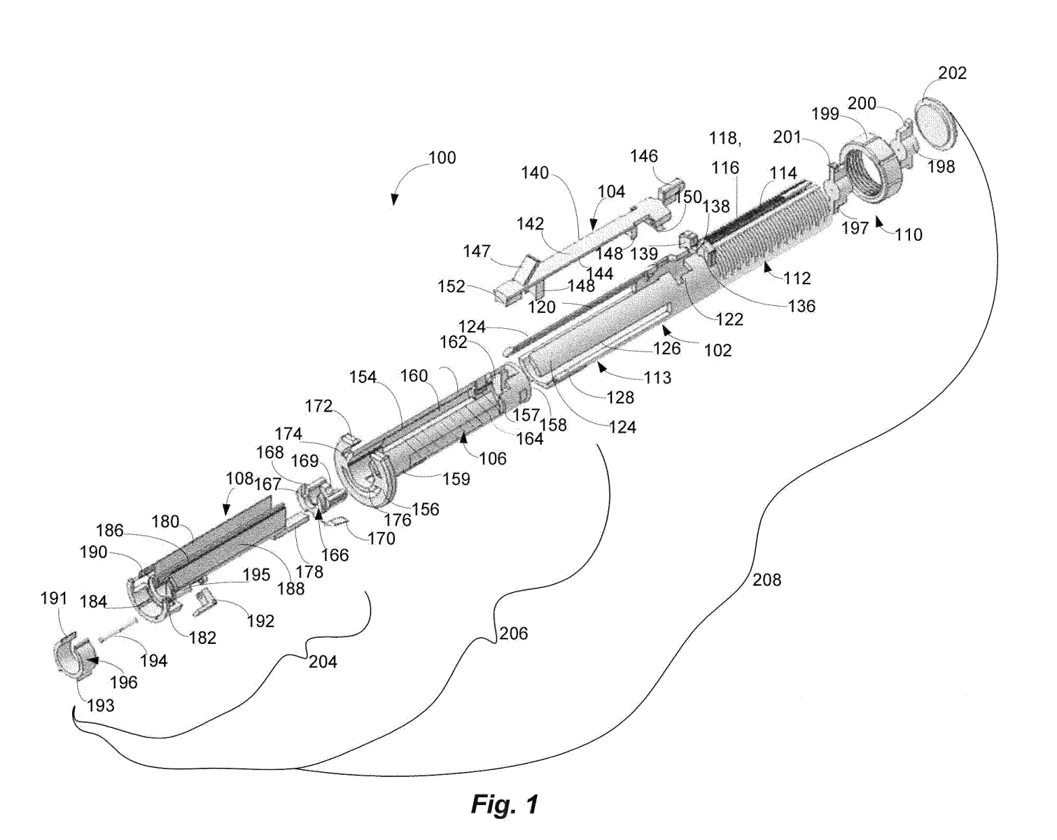

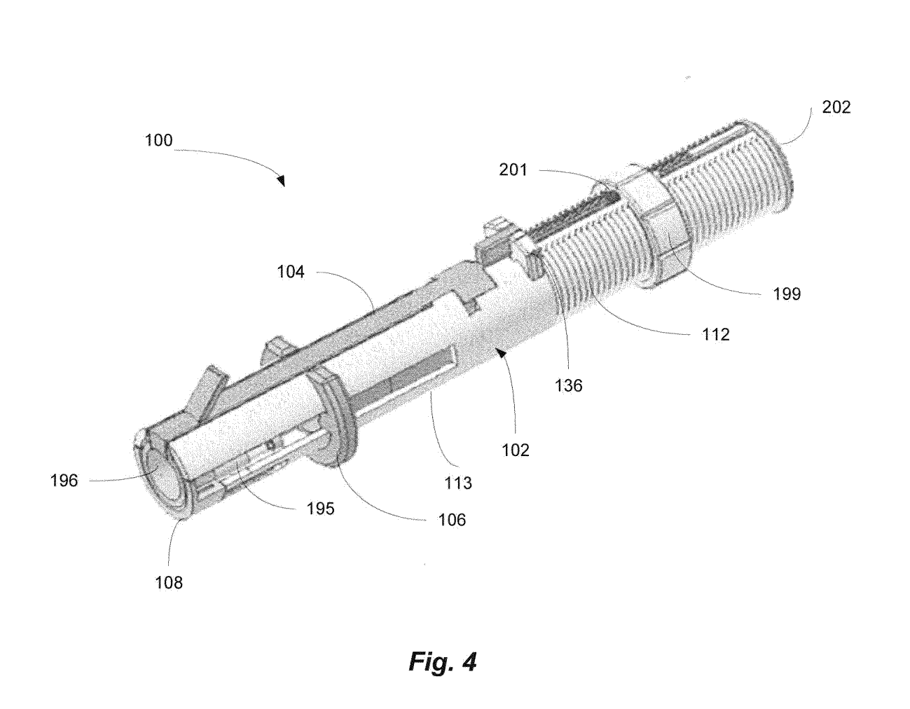

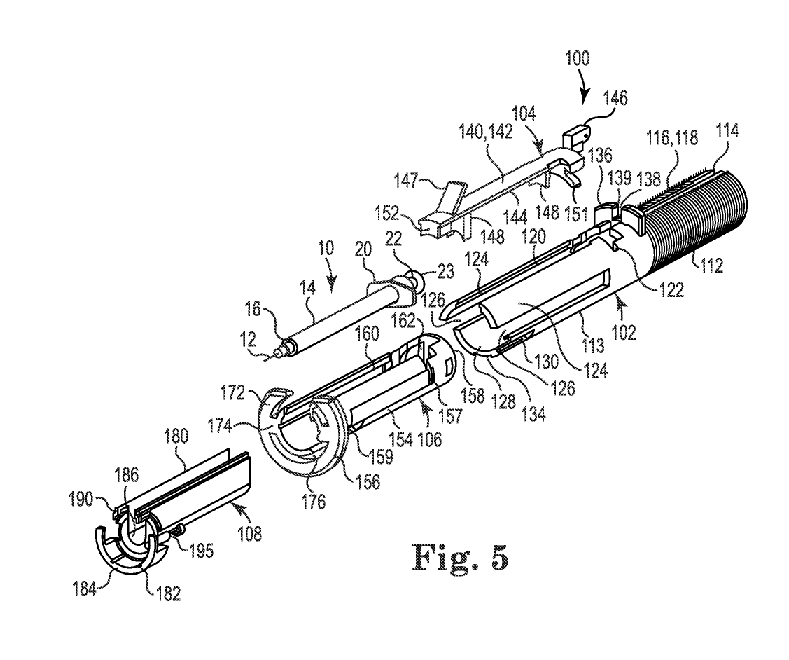

A syringe injection aid 100 of the present disclosure is illustrated in FIGS. 1-4 and is configured to interface with a standard syringe 10. The standard syringe, shown in FIG. 3, includes a needle 12, a syringe body 14 presenting a lower neck 16, side handles 20, and a syringe plunger 22 with a plunger cap 23. In utilizing the syringe injection aid 100 of the present disclosure, the user is provided with a multi-use device that enables one-handed injection as well as needle protection and needle depth control.

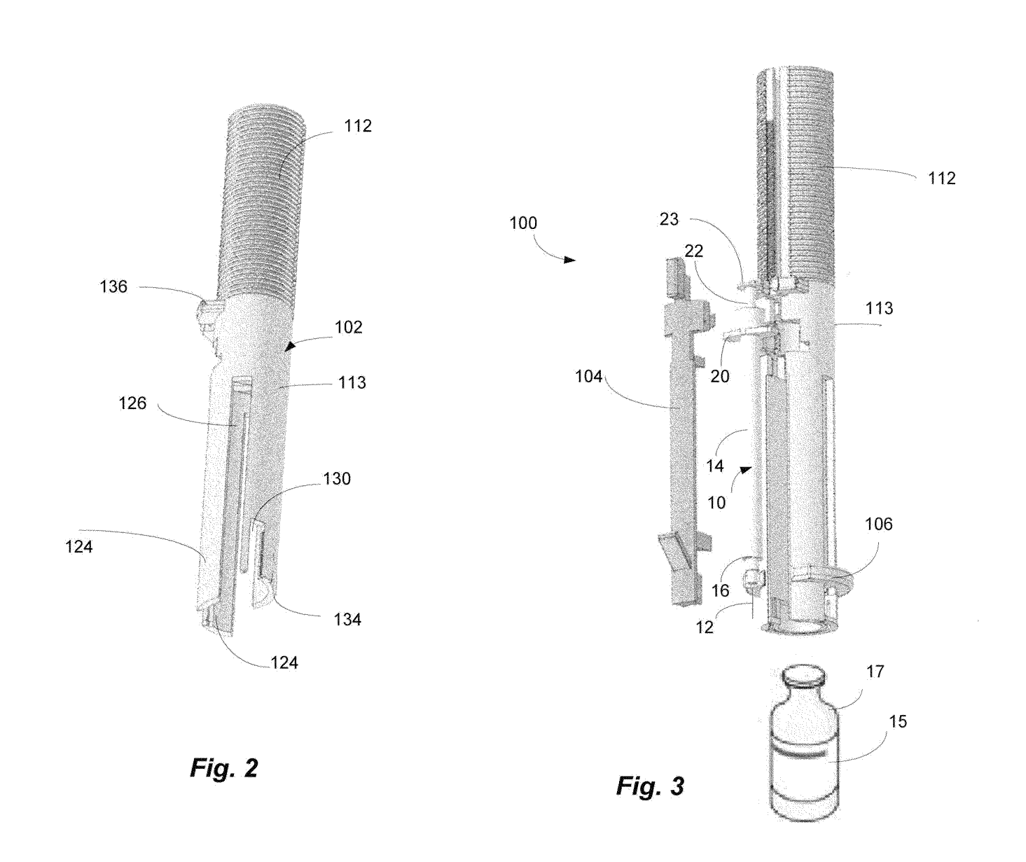

As shown, the syringe injection aid 100 generally comprises an outer shell 102, a side cap 104, a plunger 106, a tube support 108 and a gauge 110. The outer shell 102, most clearly seen in FIGS. 1 and 2, comprises a tubular configuration having a threaded upper portion 112 and a multi-slotted lower portion 113. The threaded upper portion 112 includes a centrally positioned slot 114 extending the length thereof. An inner lip 116 of the slot 114 is provided with a notched surface 118 with movement along each notch indicating a specific dosage. The lower portion 113 of the outer shell 102 is configured to receive the plunger 106 and accommodate portions of the tube support 108. Specifically, the lower portion 113 includes a centrally positioned slot 120, substantially extending the length thereof, that has been configured to accept the standard syringe 10 and thus includes a widened opening 122 designed to accommodate the side handles 20 and the plunger 22 of the syringe 10. The slot 120 is defined to either side by legs 124. The lower portion 113 further includes side slots 126 that are defined on one side by legs 124 and on the other side by rear portion 128. The rear portion 128 incorporates an indent 134 and a latch slot 130 into which a latch 192 is secureably and pivotally positioned The outer shell 102 is further defined by a side cap positioner 136 that extends outward from the lower portion 113 and is positioned intermediate the upper portion 112 and the lower portion 113. The side cap positioner 136 is defined by a central opening 138 that establishes first and second side portions 139 of the side cap positioner 136.

The side cap 104 is configured to interface with slot 120 of the lower portion 113 of the outer shell 102 and to interface with a syringe 10 held within lower portion 113. As such, the side cap 104 comprises an elongate body portion 140 having an exterior surface 142 and an interior surface 144. The exterior surface 142 is provided with a raised positioning tab 146 and a removal tab 147. The raised positioning tab 146 serves to centrally position the side cap 104 within the slot 120 and provides a snap fit to the first and second side portions 139 of the side cap positioner 136. The removal tab 147 extends angularly away from the exterior surface 142 and functions as a graspable lever by which to pivot or remove the side cap from the outer shell 102. The interior surface 144 of the side cap 104 is provided with upper and lower retention tabs 148 to assist in maintaining the position of an underlying syringe 10; the retention tabs 148 present curved forward faces to accommodate the curvature of the syringe body 14. The interior surface 144 additionally includes a ratchet stop 150. The interior surface 144 further includes a lower stabilizer 152 to stabilize the side cap 104 and assist in maintaining the side cap 104 in the slot 120; the lower stabilizer 152 also blocks access to the needle 12 of the syringe 10 when the needle 12 is in a retracted position. As such, the needle 12 is essentially confined within the syringe injection aid 100.

The plunger 106 is configured to be slidably received by the outer shell 102. The plunger 106 includes an elongate, tubular stem portion 154, an engagement flange 156, and a top cap 158. The tubular stem portion 154 includes side slot 157 which allows syringe 10 to slide by without engaging and a latch slot 159. A central slot 160, extending from the top cap 158 through the engagement flange 156, is configured to be substantially aligned with the slot 120 of the outer shell 102. The interior of the plunger 106 is provided with a slotted stop 162 proximate the top cap 158 and a slatted, interior rear surface 164, illustrated with hidden, dashed lines, to provide an interfacing surface for a ratchet 166. The ratchet 166 includes a ratchet body 168 and a catch 170, for example, a spring steel catch, ratchet arms 167, and an upper neck portion 169. The ratchet 166 is configured to accommodate the tubular configuration of the plunger 106. When the plunger 106 is received within the outer shell 102, the engagement flange 156 operates to engage the outer shell 102. In doing so, the engagement flange 156 utilizes a pair of outer arms 172, which are configured to slide along the legs 124 of outer shell 102, and a pair of intermediate arms 174, which slidably engage side slots 126 of the outer shell 102 to maintain the desired operational orientation of the plunger 106 relative to the outer shell 102. The engagement flange 156 additionally includes a curved receiving slot 176 that extends between intermediate arms 174.

The tube support 108 is configured to interface with the slidable plunger 106 and with the outer shell 102. The tube support 108 includes a centrally positioned upper tab 178, which is configured to engage the ratchet 166, a central body portion 180, and a lower tab 182 having a notched, arcuate face 184. The central body portion 180 includes an elongate central cavity 186 substantially aligned with slots 120 and 160 and elongate side walls 188 over which the tubular stem portion 154 of the plunger 106 may slide. The central body portion 180 further includes a pair of lower arm extensions 190. The tube support 108 is additionally configured to accommodate a latch trigger 194 through use of a latch/trigger receptor 195, explained further below, as well as a tube support insert 196 having alignment tabs 193 and upper engagement edges 191.

The gauge 110 includes a lower stop portion 197, an upper stop portion 198, and an adjustment ring 199. The lower stop portion 197 and upper stop portion 198 each incorporate a side wing 200 configuration with the side wing 200 of the lower stop portion 197 incorporating an indicator 201 that extends through slot 114 proximate notched surface 118 to indicate a selected dosage. The lower stop portion 197 and upper stop portion 198 are positioned to either side of the adjustment ring 199 and are connected there through by appropriate connection device, e.g., a screw. An end cap 202 may be provided to seal off the upper end of the outer shell 102.

To assemble the syringe injection aid 100, it is generally desirable to first complete the support tube assembly 204. However, it should be noted that, any assembly sequence resulting in the desired finished product is contemplated by the present disclosure. To begin the support tube assembly 204, the latch/trigger receptor 195 is fitted with a spring (not shown) and the latch trigger 194, for example, an elongate pin, is provided at each side of the spring. The tube support insert 196 is then slid into the tube support 108. Note that the alignment tabs 193 of the tube support insert 196 are accommodated by the notched arcuate face 184 of the lower tab 182 of the tube support 108 and the upper engagement edges 191 of the tube support insert 196 actively engage the pair of lower arm extensions 190 of the tube support 108. The tube support assembly 204 is now complete.

In continuing the assembly of the syringe injection aid 100, the tube support assembly 204 is incorporated into the plunger 106 to produce the plunger assembly 206. First, the ratchet 166, with the catch 170 already secured to the ratchet body 168, may be placed within the tubular stem portion 154 proximate slotted stop 162 of the plunger 106. Then central body portion 180 and upper tab 178 of the tube support are inserted into the tubular stem portion 154 of the plunger 106. As the tube support 108 is slid into the plunger 106, the lower tab 182 of the tube support 108 engages the curved receiving slot 176 of the engagement flange 156 and is slid there through until the arcuate face 184 of the lower tab 182 is flush with the engagement flange 156. The plunger assembly 206 is now complete.

Further assembly of the syringe injection aid 100 provides for incorporating the plunger assembly 206 into the outer shell 102 to establish the outer shell assembly 208. First, the latch 192 is pivotally secured within the latch slot 130. Then, the plunger assembly 206 is slid into the multi-slotted lower portion 113 of the outer shell 102. In so doing, the centrally positioned slot 120 of the lower portion 113 of the outer shell 120 is substantially aligned with the central slot 160 of the plunger 106, which is substantially aligned with the elongate central cavity 186 of the tube support 108. Further the intermediate arms 174 of the plunger 106 are engaged with the side slots 126 of the outer shell 102, the outer arms 172 of the plunger 106 are slidably positioned over the legs 124 of the outer shell 102. The plunger assembly 206 is fixedly engaged with the outer shell by engaging the lower tab 182 of the tube support 108 to the indent 134 of the outer shell 102. The engagement area at the indent 134 may be fused or glued to prevent separation of the plunger assembly 206 from the outer shell 102.

To complete the outer shell assembly 208, the lower stop portion 197 of the gauge 110 is positioned within the upper portion 112 of the outer shell 102 with the indicator 201 of the lower stop portion 197 extending visibly through the centrally positioned slot 114. The adjustment ring 199 is then threadably engaged to the threaded upper portion 113 of the outer shell 102, the upper stop portion 198 of the gauge 110 is secured to the lower stop portion 197 of the gauge 110, and the end cap 200 is secured to the upper portion 113 of the outer shell 102 to complete the outer shell assembly 208. A complete assembly of the syringe injection aid 100 finds the side cap 104 pivotably secured to the first and second side portions 139 of the side cap positioner 136.

To use the assembled syringe injection aid 100, a syringe 10, with its safety cap (not shown) in place over the needle 12, is placed within the syringe injection aid 100 such that the body 14 of the syringe 10 is substantially encompassed by the central body portion 180 of the tube support 108. Note that widened opening 122 of the outer shell 102 is configured to accept the side handles 20 of the syringe 10 to accurately position the syringe 10 within the syringe injection aid 100. At this point, the entire syringe 10, including the safety cap covered needle 12 is substantially encompassed by the syringe injection aid 100 and the side cap 104 may be pivoted downward and snapped into a closed position to complete the enclosure of the syringe 10. The upper and lower retention tabs 148 of the side cap press against the body 14 of the syringe 10 helping to maintain the position of the syringe 10.

With the empty syringe 10 loaded into the syringe injection aid 100, the adjustment ring 199 of the gauge 110 may be dialed up or down the threaded upper portion 112 of the outer shell 102 to a desired dosage indication, as indicated by a scale on the syringe injection aid 100 and by the indicator 201 of the lower stop portion 197 of the gauge 110. It should be noted that the components of the syringe injection aid 100 have been designed specific to a standard syringe, for example, a 100 unit B-D™ syringe, and thus the dosage indication on the syringe injection aid 100 has been calibrated such that operation of the syringe injection aid 100 will provide the user with the appropriate dosage. However, various embodiments of a syringe injection aid, as described herein, may be designed for any size/type syringe and the dosage indication may be calibrated accordingly.

Next, the syringe injection aid 100 may be positioned in a vertical orientation such that the end cap 202 is upon a flat surface and the needle safety cap upon the syringe needle 12 is visible to the user at the open end of the syringe injection aid. Upon removing the needle safety cap from the needle 12, the medication vial 15 may be placed upon the needle 12. Note that the tube support insert 196 and the lower stabilizer 152 of the side cap 104 essentially establish a collar configuration that is configured to fit about the neck 17 of the vial 15 and thereby center the vial 15 on the needle 12 as well as prevent over insertion of the needle into the vial. This positioning by the syringe injection aid ensures that the syringe 10 can accurately draw from the vial 15 without introducing air into the syringe 10.

Engagement of the neck 17 of the vial 15 with the syringe injection aid 100 causes engagement with the latch trigger 194 which is pushed up to causing the latch 192 to pivot in toward to the central body portion 180 of the tube support 108. Prior to this, the latch 192 extended outward from the latch slot 130 of the outer shell 102 in an orientation substantially perpendicular to the outer shell 102. In the outward orientation, the latch 192 blocks the syringe-filling pulling movement of the plunger 106 until the neck 17 of the vial 15 has engaged the syringe injection aid 100. Upon engagement, one may hold the syringe injection aid 100 and vial 15 with one hand and easily draw back the plunger 106 by grasping the engagement flange 156 to draw the medication from the vial 15 into the syringe 10. The drawing of the plunger 106 also serves to draw the plunger 22 of the syringe 10 as slotted stop 162 of the plunger 106 engages the plunger cap 23. The plunger 106 is drawn back until the top cap 158 of the plunger 106 contacts the lower stop portion 197 of the gauge 110.

The vial 15 may now be withdrawn from the syringe injection aid 100. With the needle 12 of the syringe 10 still contained within the confines of the syringe injection aid 100, the syringe injection aid 100 may then be positioned flatly against a desired injection site. Once in position, a single hand of the user may hold the syringe injection aid 100 and push the plunger 106 forward at which time the catch 170 of the ratchet 166 engages the slatted, interior back surface 164 of the plunger 106, the ratchet arms 167 engage the side handles 20 to propel the syringe body 14 forward, and slotted stop 162 of the plunger 106, which is already engaged with the plunger cap 23 of the syringe 10, may propel the plunger 22 of the syringe 10 forward.

The engagement of the ratchet arms 167 with the side handles 20 of the syringe 10 pushes the syringe body 14 forward for the length of the needle 12 of the syringe 10 as the ratchet body 168 slides along upper tab 178 of the tube support 108 until the ratchet body 166 contacts the central body portion 180 of the tube support 108. At this point, the upper tab 178 of the tube support 108 contacts the catch 170 of the ratchet 166 causing the ratchet 166 to disengage from the slatted, interior back surface 164 of the plunger 106. Any further forward movement by the ratchet 166 is stopped upon the ratchet 166 contacting the central body portion 180 of the tube support 108 which also stops any further forward movement of the syringe body 14, thus establishing an injection depth for the syringe needle 12. However, the plunger 106 continues forward pushing the plunger 22 of the syringe 10 forward to deliver the medication within the syringe 10 and complete the injection. As such, the syringe injection aid 100 operates to control the depth of penetration of the needle 12 while substantially simultaneously delivering the desired dosage of medication.

After completion of the injection, the needle safety cap may be placed over the syringe needle 12 and used to push the syringe 10 inward until the entire syringe 10 is once again within the confines of the syringe injection aid 100. Pushing the syringe 10 inward results in movement of the plunger 106 whereby the latch 192 is caused to pivot outward thereby locking the syringe injection aid 100 until another use of the syringe injection aid 100 is desired. Notably, throughout the placement of the syringe 10 within the syringe injection aid 100, the loading of the syringe 10 with medication, the delivery of the injection, and the withdrawal of the needle 12, the needle 12 may be kept within the confines of the syringe injection aid 100 preventing erroneous jabs or contamination of the needle 12. Further, with the syringe injection aid 100 in the locked position, a user may draw the side cap 104 upward and remove the syringe 10 and put a new syringe 10 in place within the syringe injection aid readying it for another use.

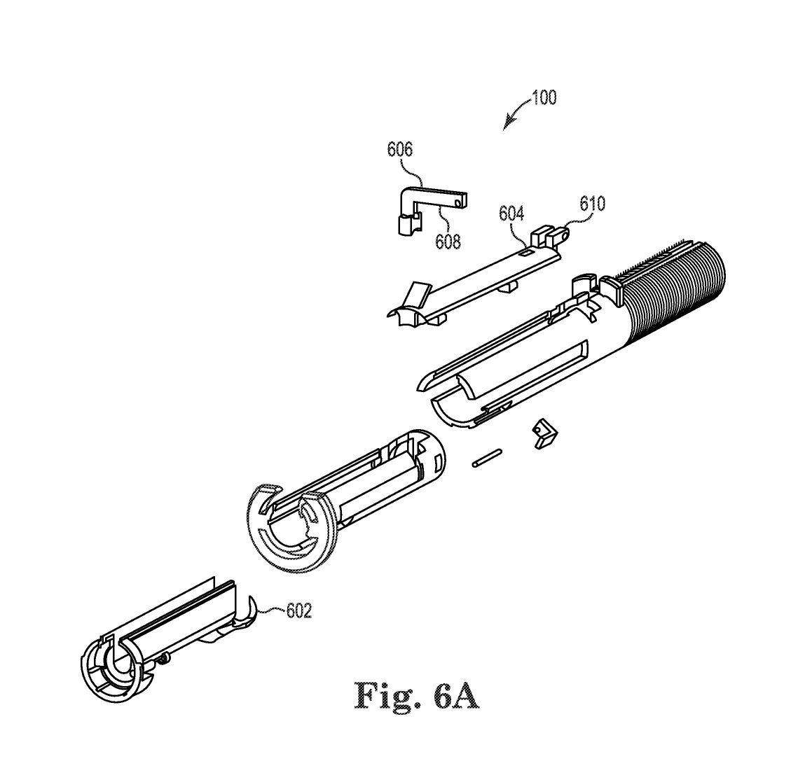

In an alternative embodiment, see FIG. 5, the ratchet 166 may be eliminated which also eliminates the need for the slatted interior surface rear surface 164 of the interior of the plunger 106. Further, the upper tab 178 of the tube support 108, whose purpose is to engage the ratchet 166, may also be eliminated. Further, the ratchet stop 150 of the side cap 104 has been replaced with arms 151 that rest behind, i.e. towards the syringe plunger 22, the side handles 20 of the syringe. Note that the tube support insert 196, latch trigger 194, spring, and latch 192 as well as gauge 110 are not shown in the illustration of FIG. 5, but should still be considered elements of the embodiment. In this configuration, the syringe plunger 22, which is engaged with the plunger 106, is used to move the body 14 of the syringe 10 forward, towards the injection site rather than the ratchet 166. The tube support 108 acts as a stop to the side handles 20 of the body 14 of the syringe 10 to prevent further forward movement of the body 14, which also serves to set the depth of needle 12 penetration at the injection site.

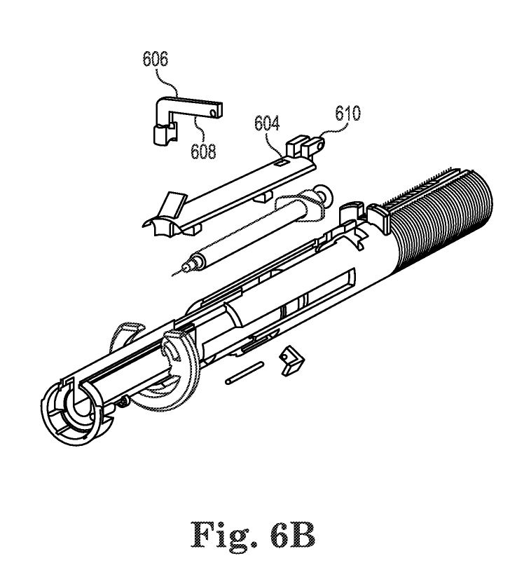

Further alternative embodiments are illustrated in FIGS. 6A-B, where FIG. 6A illustrates an exploded view of a syringe injection aid and FIG. 6B illustrates a substantially assembled syringe injection aid of FIG. 6A relative to a syringe. Here again, the ratchet 166 may be eliminated which also eliminates the need for the slatted interior surface rear surface 164 of the interior of the plunger 106. Further, the upper tab 178 of the tube support 108, whose purpose is to engage the ratchet 166, may also be eliminated. It is replaced by tab 602 having an arcuate shape for engaging curved receiving slot 176. Further, the ratchet stop 150 of the side cap 104 has been removed. Side cap 104 instead includes a slot 604 through the side cap 104 that is configured to fit a syringe retainer button 606. Syringe retainer button 606 includes an extended portion 608. The raised positioning tab 146 of FIG. 1 is replaced with a dual-armed positioning tab 610 that is configured to receive the extended portion 608 between the arms. The syringe retainer button 606 rests against a syringe 10, i.e. towards the syringe plunger 22, the side handles 20 of the syringe, to retain and immobilize the body of the syringe 10 with respect to the syringe injection aid 100. Note that the gauge 110 is not shown in the illustrations of FIGS. 6A-B, but should still be considered elements of the embodiment. Also, reference numerals for parts consistent with those labeled in FIGS. 1-4 are not repeated but should be considered the same or similar as shown in those figures. In this configuration, the syringe plunger 22, which is engaged with the plunger 106, is used to move the body 14 of the syringe 10 forward, towards the injection site rather than the ratchet 166. The tube support 108 acts as a stop to the side handles 20 of the body 14 of the syringe 10 to prevent further forward movement of the body 14, which also serves to set the depth of needle 12 penetration at the injection site.

Systems, devices or methods disclosed herein may include one or more of the features structures, methods, or combination thereof described herein. For example, a device or method may be implemented to include one or more of the features and/or processes above. It is intended that such device or method need not include all of the features and/or processes described herein, but may be implemented to include selected features and/or processes that provide useful structures and/or functionality. For example, the disclosed embodiments may be used for both human and veterinary purposes.

Various modifications and additions can be made to the disclosed embodiments discussed above. Accordingly, the scope of the present disclosure should not be limited by the particular embodiments described above, but should be defined only by the claims set forth below and equivalents thereof.

What is claimed is:

1. A syringe injection aid apparatus comprising:
a body having a lower opening defined by an actuation collar;
a syringe-interfacing plunger substantially slidably contained within the body; and
a locking mechanism pivotally secured to the body, wherein the locking mechanism is configured to prevent forward movement of the syringe-interfacing plunger when in a locked position and to enable forward movement of the syringe-interfacing plunger when in an unlocked position, wherein the unlocked position is activated by external contact with the actuation collar.

2. The apparatus of claim 1, wherein the locking mechanism comprises a spring-actuated lever.

3. The apparatus of claim 1, wherein the body includes a tube support and wherein the locking mechanism is pivotally secured to the tube support.

4. The apparatus of claim 3, wherein the tube support establishes a penetration depth of a needle of a syringe.

5. The apparatus of claim 1, wherein the body includes a hinged cap configured to centrally position a syringe body within the plunger.

6. The apparatus of claim 1, further comprising a gauge that interfaces with both the body and the plunger to establish a dosage level for a syringe.

7. A syringe injection aid apparatus comprising:
containment means including an actuation means;
syringe-interfacing means for interfacing with a syringe having a needle, the containment means for slidably containing the syringe-interfacing means; and
locking means pivotally secured to the containment means, the locking means for preventing forward movement of the syringe-interfacing means when in a locked position and for enabling forward movement of the syringe-interfacing means when in an unlocked position, wherein the actuation means for unlocking the locking means upon being externally contacted.

8. The apparatus of claim 7, wherein the locking means comprises a spring-actuated lever.

9. The apparatus of claim 7, wherein the containment means includes a tube support and wherein the locking means is pivotally secured to the tube support.

10. The apparatus of claim 9, wherein the tube support establishes a depth of penetration of the needle.

11. The apparatus of claim 7, further comprising capping means for centrally positioning the syringe within the syringe-interfacing means.

12. The apparatus of claim 7, further comprising gauge means for establishing a dosage level for the syringe by interfacing with both the containment means and the syringe-interfacing means.

13. A syringe injection aid apparatus for interfacing with a syringe having a syringe body and a needle comprising:
an outer shell;
a plunger slidably contained by the outer shell, the plunger configured to interface with the syringe and having an injection delivery position;
a tube support coupled to the outer shell, wherein the tube support includes a pivotable locking arm and an actuation collar, wherein upon external actuation of the actuation collar the locking arm is moved into an unlocked position enabling the plunger to slide past the locking arm to the injection delivery position and wherein upon retreat from the injection delivery position, the plunger causes the locking arm to move into a locked position.

14. The apparatus of claim 13, wherein the locking arm is a spring-actuated locking arm.

15. The apparatus of claim 13, wherein the tube support establishes a penetration depth of the needle of the syringe.

16. The apparatus of claim 13, further comprising a hinged cap configured to centrally position the syringe body within the plunger.

17. The apparatus of claim 13, further comprising a gauge that interfaces with both the outer shell and the plunger to establish a dosage level for the syringe.

18. A syringe injection aid apparatus comprising:
a body configured to substantially confine a syringe having a syringe plunger, a syringe body, and a needle;
a plunger substantially slidably contained within the body; and
a mechanism configured to interface with a surface of the plunger, wherein upon the mechanism engaging the surface, the mechanism engages the syringe body to move the needle a distance beyond the confines of the body and wherein upon the mechanism subsequently disengaging the surface, the plunger operates to move the syringe plunger toward the syringe body while maintaining the distance of the needle.

19. The apparatus of claim 18, wherein the mechanism comprises a ratchet.

20. The apparatus of claim 19, wherein the surface comprises an interior surface of the plunger.
